(19) United States
(12) Patent Application Publication
(10) Pub. No.: US 2016/0136203 A1
(43) Pub. Date: May 19, 2016

(54) SOMATIC STEM CELLS FOR TREATING BONE DEFECTS

(71) Applicant: StemBios Technologies, Inc., Monterey Park, CA (US)

(72) Inventor: James Wang, Monterey Park, CA (US)

(21) Appl. No.: 14/945,012

(22) Filed: Nov. 18, 2015

Related U.S. Application Data
(60) Provisional application No. 62/081,880, filed on Nov. 19, 2014.

(51) Int. Cl. A61K 35/12

(52) U.S. Cl. CPC A61K 35/12

(57) ABSTRACT

A method of treating a bone defect in a subject, comprising administering to a subject in need thereof at a bone defect site an effective amount of isolated somatic stem cells, wherein the somatic stem cells are about 2 to 8.0 μm in size and are Lgr5+ or CD349+.

### CROSS REFERENCE TO RELATED APPLICATION

This application claims priority to U.S. Provisional Application No. 62/081,880, filed on Nov. 19, 2014, the entire content of which is hereby incorporated by reference herein.

### BACKGROUND

Stem cells are pluripotent or totipotent cells that can differentiate in vivo or in vitro into many or all cell lineages. Due to their pluripotency, embryonic stem (ES) cells hold great promise for treating various diseases. Yet, ethical considerations have hampered the use of human ES cells. Stem cells of a non-embryonic origin would circumvent this obstacle. These adult stem cells have the same capability for differentiation as do ES cells.

Multipotent adult progenitor cells from bone marrow have been isolated that can differentiate into ectoderm, mesoderm and endoderm. Other types of cells, including marrow-isolated adult multi-lineage inducible cells and single cell clones derived from bone marrow also have the same multi-potential ability for differentiation. Such multipotent somatic cells are difficult to obtain, culture, and expand.

### SUMMARY

Described herein is a method of treating a bone defect in a subject. The method includes administering to a subject in need thereof at a bone defect site an effective amount of isolated somatic stem cells. The somatic stem cells are about 2 to 8.0 μm in size and are Lgr5+ or CD349+.

The isolated somatic stem cells can be obtained by the following procedure: incubating a sample from a donor subject with EDTA or heparin in a container until the sample is separated into an upper layer and a lower layer; collecting the upper layer; and isolating from the upper layer a population of somatic stem cells that are about 2 to 8.0 μm in size and are Lgr5+ or CD349+.

The details of one or more embodiments are set forth in the accompanying drawing and the description below. Other features, objects, and advantages of the embodiments will be apparent from the description and drawing, and from the claims.

### BRIEF DESCRIPTION OF DRAWINGS

FIG. 1 is a set of images that demonstrate repair of a cranial defect using SB cells. (A): positive and negative controls. (B): SB cells.

### DETAILED DESCRIPTION

It was unexpectedly discovered that small adult stem cells, i.e., SB cells, can be isolated from samples from subjects. SB cells are pluripotent or totipotent stem cells that can differentiate into cell types associated with the three embryonic germ layers, namely, ectoderm, endoderm, and mesoderm. See US2012/0034194.

SB cells isolated from a biological sample (e.g., a bone marrow sample) are about 2 to 6.0 μm in size, CD133−, CD34−, CD90−, CD66e−, CD31−, Lin1−, CD61−, Oct4+, Nanog+, and Sox2−. Among the SB cell population, there is a unique subpopulation of cells that are CD9− and Lgr5+ (“Lgr5+ SB cells”). There is another subpopulation of SB cells that are CD9+ and CD349+ (“CD349+ SB cells”).

SB cells can be isolated from a sample using the following procedure. The sample is incubated with EDTA or heparin in a container (e.g., in an EDTA tube) until the sample separates into an upper layer and a lower layer. The incubation can be performed for 6 to 48 hours at 4° C. The upper layer produced by the above incubating step contains SB cells (e.g., Lgr5+ SB cells and CD349+ SB cells), which can be isolated using methods based on cell size (e.g., centrifuging and filtering) or those based on cell surface markers (e.g., flow cytometry, antibodies, and magnetic sorting).

To enrich SB cells, Lin+ cells and CD61+ cells can be removed from the cell population in the upper layer. Alternatively, Lin− cells and CD61− cells can be selected from the cell population. Lin+ and CD61+ cells can be removed or selected using methods known in the art, e.g., EasySep Biotin Selection Kit and EasySep PE Selection Kit.

To further enrich SB cells, granulocyte-colony stimulating factor (GCSF) or fucoidan can be administered to a subject before a sample is obtained from the subject. For example, the subject can be injected with 5μg/kg/per day of GCSF for 1 to 5 days prior to obtaining the sample. Data described below show that GCSF can mobilize SB cells. GCSF-mobilized SB cells are slightly larger in size, i.e., about 4 to 8 μm.

SB cells can be isolated from a sample such as a blood, bone marrow, skeletal muscle, or adipose tissue sample. In an embodiment where the sample is a skeletal muscle or adipose tissue sample, prior to the incubating step, the tissue sample can be first digested with a collagenase to release individual cells from the extracellular matrix. The sample can be obtained from a human subject.

Isolated SB cells, Lgr5+ SB cells, or CD349+ SB cells can be further propagated in a non-differentiating medium for more than 10, 20, 50, or 100 population doublings without indications of spontaneous differentiation, senescence, morphological changes, increased growth rate, or changes in ability to differentiate. These stem cells can be stored by standard methods before use.

The term “stem cell” refers to a cell that is totipotent or pluripotent, i.e., capable of differentiating into a number of final, differentiated cell types. Totipotent stem cells typically have the capacity to develop into any cell type. Totipotent stem cells can be embryonic or non-embryonic in origin. Pluripotent cells are typically cells capable of differentiating into several different, final differentiated cell types. Unipotent stem cells can produce only one cell type, but have the property of self-renewal which distinguishes them from non-stem cells. These stem cells can originate from various tissue or organ systems, including blood, nerve, muscle, skin, gut, bone, kidney, liver, pancreas, thymus, and the like.

The stem cells disclosed herein are substantially pure. The term “substantially pure”, when used in reference to stem cells or cells derived there from (e.g., differentiated cells), means that the specified cells constitute the majority of cells in the preparation (i.e., more than 20%, 30%, 40%, 50%, 60%, 70%, 80%, 90%, or 95%). Generally, a substantially purified population of cells constitutes at least about 70% of the cells in a preparation, usually about 80% of the cells in a preparation, and particularly at least about 90% of the cells in a preparation (e.g., 95%, 97%, 99% or 100%).

The terms “proliferation” and “expansion,” as used interchangeably herein with reference to cells, refer to an increase in the number of cells of the same type by division. The term “differentiation” refers to a developmental process whereby cells become specialized for a particular function, for example, where cells acquire one or more morphological characteristics and/or functions different from that of the initial cell type. The term “differentiation” includes both lineage commitment and terminal differentiation processes. Differentiation may be assessed, for example, by monitoring the presence or absence of lineage markers, using immunohistochemistry or other procedures known to a worker skilled in the art. Differentiated progeny cells derived from progenitor cells may be, but are not necessarily, related to the same germ layer or tissue as the source tissue of the stem cells. For example, neural progenitor cells and muscle progenitor cells can differentiate into hematopoietic cell lineages.

The terms “lineage commitment” and “specification,” as used interchangeably herein, refer to the process a stem cell undergoes in which the stem cell gives rise to a progenitor cell committed to forming a particular limited range of differentiated cell types. Committed progenitor cells are often capable of self-renewal or cell division.

The term “terminal differentiation” refers to the final differentiation of a cell into a mature, fully differentiated cell. For example, neural progenitor cells and muscle progenitor cells can differentiate into hematopoietic cell lineages, terminal differentiation of which leads to mature blood cells of a specific cell type. Usually, terminal differentiation is associated with withdrawal from the cell cycle and cessation of proliferation. The term “progenitor cell,” as used herein, refers to a cell that is committed to a particular cell lineage, which gives rise to cells of this lineage by a series of cell divisions. An example of a progenitor cell would be a myoblast, which is capable of differentiation to only one type of cell, but is itself not fully mature or fully differentiated.

Lgr5+ or CD349+ SB cells can be used to treat or repair a bone defect in a patient. To treat a bone defect in a patient, Lgr5+ or CD349+ SB cells alone can be administered to the subject at the defect site. The cells can also be administered together with a bone graft (e.g., an autograft or allograft) or a bone graft substitute (e.g., demineralized bone matrix, collagen-based matrix, hydroxyapatite, calcium phosphate, and calcium sulfate).

Lgr5+ or CD349+ SB cells can also be first implanted in a scaffold or matrix. The scaffold or matrix can then be implanted at the defect site. Stem cell scaffolds composed of one or more materials (e.g., collagen, agarose, alginate, hyaluronan, chitosan, PLGA, and PEG) are known in the art.

A “bone defect” refers to a lack or deficiency of bone tissue (i.e., the mineralized matrix of a bone) in an area in a bone. A bone defect can result from various causes, such as trauma, cancer, or a congenital condition.

Both heterologous and autologous Lgr5+ or CD349+ SB cells can be used to treat a patient. If heterologous cells are used, HLA-matching should be conducted to avoid or minimize host reactions. Autologous cells can be enriched and purified from a subject and stored for later use. The cells may be cultured in the presence of host or graft T cells ex vivo and re-introduced into the host. This may have the advantage of the host recognizing the cells as self and better providing reduction in T cell activity.

Genetically engineered histocompatible universal donor Lgr5+ or CD349+ SB cells can also be prepared using methods known in the art. More specifically, the stem cells described herein can be genetically engineered to not express on their surface class II MHC molecules. The cells can also be engineered to not express substantially all cell surface class I and class II MHC molecules. As used herein, the term “not express” means either that an insufficient amount is expressed on the surface of the cell to elicit a response or that the protein that is expressed is deficient and therefore does not elicit a response.

“Treating” refers to administration of a composition (e.g., a cell composition) to a subject, who is suffering from or is at risk for developing that disorder, with the purpose to cure, alleviate, relieve, remedy, delay the onset of, prevent, or ameliorate the disorder, the symptom of the disorder, the disease state secondary to the disorder, or the predisposition toward the damage/disorder. An “effective amount” refers to an amount of the composition that is capable of producing a medically desirable result in a treated subject. The treatment method can be performed alone or in conjunction with other drugs or therapies.

The specific example below is to be construed as merely illustrative, and not limitative of the remainder of the disclosure in any way whatsoever. Without further elaboration, it is believed that one skilled in the art can, based on the description herein, utilize the present disclosure to its fullest extent. All publications cited herein are hereby incorporated by reference in their entirety.

### EXAMPLE

A bone marrow sample was drawn from a human subject and placed in an anti-clotting EDTA tube. After incubating the tube for 6 to 48 hours at 4° C., the sample separated into two layers. The top layer contained a somatic stem cell population (SB cells), which was further analyzed by C6 accuri flow cytometry, immunocytochemistry, and RT-PCR. The bottom layer contained red and white blood cells, which are not smaller than 6.0 μm.

Using sizing beads to run the flow cytometry, the size of the SB cells was determined. The size of the SB cells was between 2 and 6 microns. SB cells were either Lgr5+ or CD349+. Lgr5 was expressed by 32% of the cell population in gate P2.

We found that SB cells can be mobilized by injection of GCSF. The same human subject was injection with 5 μg/kg/per day of GCSF for 5 days. A peripheral blood sample was collected about 3.5 hours after the last injection. SB cells were isolated from the blood sample as described above and analyzed by flow cytometry. As compared to SB cells isolated from the subject prior to the GCSF injection, the cell size increased to 4-8 micron and the percentage of Lgr5+ cells also increased.

Normal human blood (purchased from AllCell) was placed in an anti-clotting EDTA tube, to which HetaStarch (purchased from StemCell) was added. The blood sample separated into two layers. CD61+ platelets and Lin+ cells, which include red and white blood cells, were removed from the top layer using the EasySep Biotin Selection Kit and EasySep PE Selection Kit, respectively, following the manufacturer's instructions. After Lin+ and CD61+ cells were removed, a purified population of Lgr5+ or CD349+ SB cells was obtained.

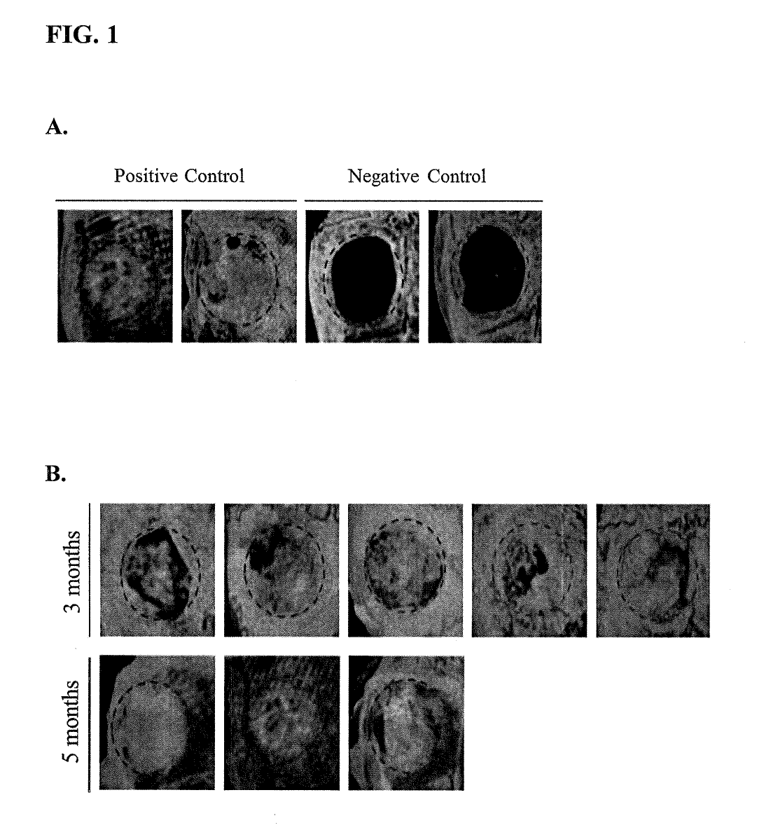

1 million of the above purified SB cells together with collagen sponges were engrafted into a SCID mouse at a cranial defect site created by surgical removal of a section of the bone from the skull. The mouse was analyzed by microcomputed tomography images 3 or 5 months after engraftment of the SB cells in the defected site. As shown in FIG. 1, SB cells were able to form bone structures to repair the defect site. A mouse treated with human bone marrow cells that overexpress a human bone morphogenetic protein 7 (hBMP7) was used as a positive control. A mouse treated with collagen sponges and PBS only was used as a negative control.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the described embodiments, and without departing from the spirit and scope thereof, can make various changes and modifications of the embodiments to adapt it to various usages and conditions. Thus, other embodiments are also within the claims.

1. A method of treating a bone defect in a subject, comprising administering to a subject in need thereof at a bone defect site an effective amount of isolated somatic stem cells, wherein the somatic stem cells are 2 to 8.0 μm in size and are Lgr5+ or CD349+.

2. The method of claim 1, wherein the somatic stem cells are CD133−, CD34−, CD90−, CD66e−, CD31−, Lin1−, CD61−, Oct4+, Nanog+, and Sox2−.

3. The method of claim 1, wherein the somatic stem cells are Lgr5+.

4. The method of claim 1, further comprising administering a bone graft or bone graft substitute to the bone defect site.

5. The method of claim 1, wherein the somatic stem cells are implanted in a scaffold.

6. The method of claim 1, wherein the somatic stem cells are obtained by the following procedure:
incubating a sample from a donor subject with EDTA or heparin in a container until the sample is separated into an upper layer and a lower layer,
collecting the upper layer, and
isolating from the upper layer a population of somatic stem cells that are 2 to 8.0 μm in size and are Lgr5+ or CD349+.

7. The method of claim 6, wherein the sample is a blood or bone marrow sample.

8. The method of claim 6, wherein, prior to obtaining the sample from the donor subject, the donor subject is administered with a granulocyte-colony stimulating factor or fucoidan.

9. The method of claim 1, wherein the somatic stem cells are autologous or heterologous to the subject.

10. The method of claim 1, further comprising, prior to the administering step:
incubating a sample from a donor subject with EDTA or heparin in a container until the sample is separated into an upper layer and a lower layer,
collecting the upper layer, and
isolating from the upper layer a population of somatic stem cells that are about 2 to 8.0 μm in size and are Lgr5+ or CD349+.

11. The method of claim 10, further comprising removing Lin+ cells and CD61+ cells from the upper layer.

12. The method of claim 10, wherein, before the sample is obtained from the donor subject, the donor subject is administered with a granulocyte-colony stimulating factor or fucoidan.

13. The method of claim 12, wherein the donor subject is the subject with a bone defect or another subject.

14. The method of claim 10, wherein the sample is a blood or bone marrow sample.
